(19) United States
(12) Patent Application Publication
(10) Pub. No.: US 2024/0415887 A1
(43) Pub. Date: Dec. 19, 2024

(54) TERMINAL EFFECTOR T CELLS, PROCESS FOR THEIR PRODUCTION AND THEIR ISOLATION AND THEIR THERAPEUTIC USE

(71) Applicants: UNIVERSITÉ GRENOBLE ALPES, Saint Martin D'heres (FR); INSTITUT NATIONAL DE RECHERCHE POUR L'AGRICULTURE, L'ALIMENTATION ET L'ENVIRONNEMENT, Paris (FR)

(72) Inventors: Yahia CHEBLOUNE, Bourgoin Jallieu (FR); Sara EL KENNANI, Saint-Égrève (FR)

(73) Assignees: UNIVERSITÉ GRENOBLE ALPES, Saint Martin D'heres (FR); INSTITUT NATIONAL DE RECHERCHE POUR L'AGRICULTURE, L'ALIMENTATION ET L'ENVIRONNEMENT, Paris (FR)

(21) Appl. No.: 18/703,236

(22) PCT Filed: Oct. 19, 2022
(86) PCT No.: PCT/EP2022/079085

§ 371 Date: Apr. 19, 2024

(30) Foreign Application Priority Data
Oct. 19, 2021 (FR) 21/11103

(51) Int. Cl. A61K 35/17; A61K 9/00; A61K 39/00; A61P 31/16; B01D 15/38; C12N 5/0783

(52) U.S. Cl. CPC A61K 35/17; A61K 9/0019; A61K 39/4611; A61K 39/464838; A61P 31/16; B01D 15/3809; C12N 5/0636; A61K 2239/31

(57) ABSTRACT

An in vitro or ex vivo method for producing and isolating a cell subpopulation including T cells specific for an antigen linked to a disease of interest, which includes steps of obtaining, from an isolated human biological sample, a population of mononuclear cells including T cells specific for the antigen with a high proliferative capacity, culturing these mononuclear cells in a suitable cell culture medium containing the antigen, and isolating T cells specific to the antigen which do not express the CD45RO and CD27 markers at their surface. The cell subpopulation thus obtained and isolated finds application in particular for the treatment of the disease of interest.

The present invention relates to the field of cell therapy against infectious and chronic pathologies, in particular severe infectious diseases such as Covid-19.

More particularly, the present invention relates to a method for the production and isolation of a cell subpopulation comprising terminal effector T cells specific for an antigen linked to a disease of interest, as well as the cell subpopulation obtained by such a method. The invention also relates to the therapeutic use of such a cell subpopulation, and to a pharmaceutical composition containing it.

The emergence of the novel human coronavirus SARS-COV-2 in the fall of 2019 and the expansion of its epidemic into a global pandemic has revealed the need to have emergency therapeutic solutions in order to rapidly halt the progression of the pandemic in the absence of a vaccine.

Several treatments used against infectious and/or chronic diseases have been repositioned for taking care of Covid-19 patients, as described in particular in the publication by Mashouri et al., 2021, Biochem Pharmacol. 183: 114296. For example, these consist of chloroquine and hydroxychloroquine, azithromycin, omeprazole, ivermectin, etc. However, clinical trials conducted in many countries on infected patients have not been conclusive. The antiviral molecules known to be inhibitors of viral RNA polymerase, such as remdesivir, ribovirin or favipiravir, have also been tested without success. Other viral protease inhibitor molecules, such as dolutegravir, nelfinavir or lopinavir, have not shown therapeutic effects against Covid-19. Finally, inhibitors of endocytosis and of the fusion between the viral and cellular membrane, such as arbidol and hydrochloroquine, have also been tested, but without success.

As a result, neither repositioning nor development of molecules have showed convincing efficacy against Covid-19.

The transfer of plasma from convalescent donors who have controlled and eliminated the infection by SARS-COV-2 has been one of the great first emergency treatment hopes, as described in particular in the publication by Ghareeb et al. 2021, J Pharm. Investig. p1-16. The innocuousness and efficacy of the Covid-19 convalescent plasma have first been tested in the treatment of patients with low to average symptoms of Covid-19 arriving at emergency services, in order to prevent the severity of this disease caused by SARS-COV-2. Subsequently, the health authorities of the United States have issued an emergency use authorization to treat hospitalized patients suffering from Covid-19 with convalescent plasma from people who have recovered from the virus. However, this therapeutic hope has vanished for several reasons: the amount of plasma available is insufficient; the neutralizing antibody titers in this plasma are low; a large number of convalescent persons lose the neutralizing antibodies very rapidly.

Finally, treatments based on artificially produced monoclonal antibodies have been associated with encouraging results. In this respect, mention may be made of bamlanivimab and REGN-CoV, monoclonal antibodies produced respectively by the companies Eli Lilly and Regenerated. These antibodies are designed to attach directly on the spicules of the virus and block their interaction with the cellular receptors, so as to abolish the infection. However, they are very expensive and therefore not very accessible.

Several studies show that upon infection with SARS-COV-2, the reactivity of the T lymphocytes, involved in the cellular immune response, seems to be determinant for the control of the progression of the disease (Yang et al, 2021, Clin. Transl. Immunol. 10(3): E1259). Tests of cellular therapies have thus been carried out aimed at stimulating cellular immune responses.

The first strategy proposed by the prior art relates to the use of stem cells derived from cord blood or placenta as sources capable of stimulating immunity in infected patients.

The second strategy relates to the reimplantation of autologous T cells of the patient (CAR/TCR-T). This technology consists in modifying ex vivo the T cells of the patient using the chimeric antigen receptor. These modified T cells specifically recognize the cells expressing the antigens and eradicate them by cytolysis. However, besides the complexity of set-up of this technology for a wide-scale application, it is also associated with very considerably side effects represented essentially by cytokine release syndrome, which corresponds to a severe reaction of the immune responses.

Moreover, interest has been shown on the use of the T cells of the immunity of convalescent donors. Indeed, several studies have revealed that unlike neutralizing antibodies which tend to disappear rapidly in the peripheral blood in the convalescent patients of Covid-19, the T cells specific to the virus persist for a longer time and can be amplified in in vitro culture. However, the use of the T cells specific to immune adoptive transfer virus encounters the problems associated with side effects associated with graft versus host (“Graft versus host”: GVH) immune responses syndrome, leading to very severe diseases. To expect a chance of success, the donors and the recipients must possess a prior compatibility of their human leukocyte antigens (HLA). This considerably limits and complicates the therapeutic process.

The publication by Cooper et al., in Frontier in Immunology, 2021, 11, Article 598402, describes a population of human T cells specific for SARS-COV-2 derived from convalescent donors, comprising memory effector terminal T cells expressing the marker CD45RA (TEMRA). The publication by Neidleman et al, in Cell reports Medicine, 2020, 1: 100081, describes a cell population containing TEMRA.

The present invention aims to propose a solution for the therapeutic treatment of Covid-19, and more generally infectious diseases, in particular viral diseases, and/or chronic diseases, which has good efficacy, which is simple and rapid to implement, and which is not associated with side effects that can lead to the death of the patient, in particular diseases of the graft rejection type and of the graft versus host immune responses.

To this end, the present Inventors have focused on a particular type of T cells, more specifically the effector T cells with a terminal differentiation, also called “terminal effector T cells”. These cells, which are characterized in particular by a very short lifetime, in the range of a few days, more specifically less than or equal to five days, will be designated in the present description, for more convenience, by the acronym SLECs (standing for Short-Lived Effector T Cells). SLECs are distinguished in particular from TEMRAs by the absence of the expression of the CD45RO marker at their surface. While TEMRAs are CD45RA⁺/CD45RO^{lo}/CD27⁻, SLECs are CD45RA⁺/CD45RO⁻/CD27⁻. In particular, SLECs are fitted with the highest effector functions and the shortest lifetime compared to all other types of cells, including TEMRAs.

The present Inventors have discovered that, after having been treated in vitro in a specific manner, such disease-specific cells are able, thanks to their cytotoxicity, to recognize the cells affected by this disease and to remove them rapidly from the organism. In particular, it has been discovered by the present Inventors that: more than 95% of such CD8+ type human SLECs consist of granzyme B+ and more than 40% of them also secrete IFN-gamma; and more than 50% of such CD4+ type human SLECs consist of granzyme B+ and more than 40% of them produce IFN-gamma. Thus, such SLECs have all of the characteristics of cytotoxic effector cells armed to eradicate the infected cells, so that the use of such cells in a curative cell therapy treatment allows effectively fighting the disease, and that being so in a very unexpected manner given the short lifetime which characterizes them, in the range of 3 to 5 days in cell culture. Moreover, this short lifetime advantageously avoids, after introduction of these cells into the organism, undesirable side effects such as rejection reactions known as graft versus host disease. These specific T cells are also simple and rapid to produce and purify, so that they constitute an easy access therapeutic active agent.

According to a first aspect, the present invention relates to an in vitro or ex vivo method for producing and isolating a cell subpopulation comprising T cells specific to an antigen linked to a disease of interest, more specifically, a population of cells comprising terminal effector T cells (SLECs) characterized by a cytotoxic character and a short lifetime, specific for this antigen.

This method includes steps of:
a/ starting from an isolated human biological sample, obtaining a population of mononuclear cells comprising T cells specific for said antigen with a high proliferative capacity,
b/ culturing said mononuclear cells for 24 to 72 hours in a suitable cell culture medium containing said antigen, so as to cause the differentiation of the effector T cells with memory, to produce terminal effector T cells, preferably under conditions allowing maximizing the production of these,
and c/ isolating from the culture medium the T cell that do not express at their surface the markers CD45RO and CD27.

These T cells specific to the antigen which do not express the markers CD45RO and CD27 at their surface are very predominantly SLECs, a large proportion of which are specifically directed against the pathological cells associated with the disease of interest. Since the effector T cells secreting cytokines and accumulating the lytic molecules (Granzyme B) which result from an in vitro antigenic re-stimulation are predominantly specific to the antigen, the cell subpopulation obtained upon completion of the isolation step c/, complying with the phenotype (CD45RO−/CD27−) is composed of at least 50%, and even at least 60% (in number) of SLECs specific to the antigen. In particular implementations of the invention wherein the method comprises a step of purification of the antigen-specific cells, carried out, in a conventional manner, before or after step c/, the cell subpopulation obtained upon completion of the method may contain at least 90% of SLECs specific to the antigen, or consist of, that is to say contain only SLECs specific to the antigen. When administered to a subject with the disease of interest, in the context of a treatment of the disease by cell therapy, such cells have the ability to recognize the pathological cells and to remove them rapidly from the organism. Furthermore, they do not cause any undesirable reaction, in particular of the type of rejection of the graft, likely to cause its death. Furthermore, repeated administration of such cells to an individual has no effect of inducing the apparition of the associated disease, which advantageously allows considering use thereof for the treatment of chronic diseases.

The method according to the invention is advantageously simple and rapid to implement.

It may comprise a prior step of identifying, for a targeted disease of interest, an antigen linked to this disease, against which the T cells produced in the organism will be specifically directed, and which will be used in the context of the method, for the cell culture step b/. For such an identification, a person skilled in the art could refer to the literature published on the subject, or proceed empirically.

For example, for the Covid-19 disease, an associated antigen is, as indicated hereinabove, the SARS-COV-2 virus; the H1N1 virus is an antigen associated with influenza; the basal myelin protein is an antigen associated with multiple sclerosis; in the context of cancers, for example neoantigens associated with tumor development or tumor-specific antigens (TSA) are also known. In the case of autoimmunity, overexpression of free antigens occurs and/or associated with the cells that trigger immune reactions.

Step a/ of obtaining a population of mononuclear cells comprising T cells specific to the antigen with a high proliferative capacity may be carried out in any manner known to a person skilled in the art.

In the present description, by “T cells with a high proliferative capacity”, it should be understood, in a conventional manner per se, T cells that are capable of performing at least one to two divisions in 24 hours. In particular, such T cells consist of terminal differentiation effector T cells and effector T cells with memory.

In particular embodiments of the invention, step a/ of obtaining a population of mononuclear cells comprising T cells specific to the antigen, with a high proliferative capacity, comprises the identification/selection of a human adult peripheral blood biological sample, isolated from the human body, comprising a population of T cells specific to the antigen with a high proliferative capacity, and the isolation of mononuclear cells from this biological sample.

The identification of a human peripheral blood biological sample comprising a population of T cells specific to the targeted antigen and with a high proliferative capacity may be carried out in any conventional manner. After having provided a human peripheral blood sample isolated from the human body, a person skilled in the art can in particular proceed with tests primarily intended to determine whether this sample comprises a population of T cells specific to the antigen, then whether these T cells have, or not, a strong proliferative capacity. To this end, any conventional methods known to a person skilled in the art may be implemented.

For example, to determine whether a human peripheral blood sample isolated from the given human body contains, or not, a population of T cells specific to the target antigen, the immunoenzymatic technique known under the name Elispot (standing for “enzyme-linked immunospot”), which is a standard method for measuring the proportion of effector cells in a heterogeneous population of cells, may be implemented. Schematically, this method consists in depositing, in wells of multi-well plates at the surfaces of which antibodies specific for cytokines (IL-2, IFN-γ or TNF-α for example) are fixed, the sample to be analyzed in mixture with the target antigen. In the presence of T cells specific to this antigen in the sample, a secretion of cytokines is then produced, which are captured by the antibodies present in the wells. The spots thus formed indicate the presence in the sample of effector cells specific to the studied antigen, the proportion of these cells in the sample could even be quantified. It is within a person skilled in the art to determine the exact operating conditions for implementing this Elispot technique for each human peripheral blood sample and each specific targeted antigen.

In order to determine whether the proliferative capacity of at least some of these T cells specific to the target antigen present in the sample is or is not strong enough to be able to use the sample for the remainder of the method according to the invention, a person skilled in the art can implement any conventional method for determining the cell division rate of a cell. For example, this division rate may be measured either by a commercially-available proliferation test, or by marking the cells with a vital dye, in particular the carboxyfluorescein succinimidyl ester (CFSE, standing for Carboxyfluorescein succinimidyl ester), followed by flow cytometry of the dilution of the dye following the cell divisions taking place.

The step of isolating peripheral blood mononuclear cells (designated by the abbreviation PBMCs, standing for Peripheral Blood Mononuclear Cells) from the human peripheral blood sample identified as comprising a population of T cells specific to the antigen with a high proliferative capacity, that is to say identified as suitable to serve as a basis for implementing the steps of the method according to the invention, may be implemented in any manner known to a person skilled in the art, for example by centrifuging a human peripheral blood sample collected on ethylenediaminetetraacetic acid (EDTA 5 mM) in order to avoid coagulation thereof, and recovering the obtained cell pellet.

The population of mononuclear cells thus isolated obtained upon completion of this isolation step typically contains effector T cells with memory specific to the antigen.

In preferred implementations of the invention, the human peripheral blood biological sample is a peripheral blood sample of a human individual having been affected at least once by the disease of interest, and preferably being in the convalescence phase, that is to say a few days to a few months after the cessation of the symptoms of the disease. Such a sample is highly likely to contain a high proportion of T cells specific to the disease associated with the disease and with a high proliferative capacity, having been produced by the individual in response to the contraction of the disease.

In other words, the human peripheral blood biological sample may for example consist of a sample of peripheral blood of a human individual having been immunized against the disease, by vaccination (this individual could also, where appropriate, have contracted the disease before and/or after administration of the vaccine).

In alternative embodiments of the invention, step a/ of obtaining a population of mononuclear cells comprising T cells specific to the antigen with a high proliferative capacity may be carried out in vitro from of an umbilical cord blood biological sample of any individual, that is to say of an individual having not necessarily contracted the disease and/or being immunized against the disease. This step then comprises, carried out in a conventional manner, obtaining, from such a sample, dendritic cells adherent to the plastic and culture thereof with an antigen corresponding to the target disease, according to a protocol described in the literature (Abbasian et al., Immunobiology, 2006, 108: 203; Bedke et al., J. Immunol. Methods, 2020, 477: 112703). These cells present the antigen on their surface by major histocompatibility complex (MHC). The co-culture of these cells with the non-adherent cells extracted from the same sample, containing the T lymphocytes, then allows obtaining a population of mononuclear cells comprising T cells specific to the antigen with a high proliferative and differentiation capacity, capable of generating terminal effector cells in step b/ of the method according to the invention.

Such a mode for obtaining a population of mononuclear cells comprising T cells specific to the antigen with a high proliferative capacity is particularly suitable when the targeted disease is not a viral infectious disease, but a chronic disease, a cancer or an autoimmune disease, for which the peripheral blood of the convalescent individuals includes no or few T cells with a high proliferative capacity.

Step b/ of the method according to the invention, of culture of the mononuclear cells obtained from the biological sample, in particular from a human peripheral blood biological sample, in the presence of the antigen, advantageously allows producing SLECs, a predominant proportion of which are specific to this antigen, by in vitro differentiation of the effector T cells with a memory present in the population of implemented mononuclear cells.

It is within the skills of a person skilled in the art to select a medium cell culture suitable for this culture. As such a culture medium, mention may be made, for example, of the commercial medium RPMI 1640 (Roswell Park Memorial Institute), in particular supplemented with 10% of fetal calf serum, 1% penicillin/streptomycin solution, 1% HEPES buffer and 1% glutamine. Another example of such a cell culture medium is the serum-free medium commercialized under the name AIM-V® by the company Gibco.

The cells may be cultured in an incubator for cell culture, preferably at 37° C., and preferably under 5% of CO₂ and/or a saturating humidity.

The concentration of antigens to be applied in the cell culture medium depends on the particular antigen implemented. It is upon the person skilled in the art to determine this concentration, so as to maximize the amount of antigen-specific SLECs produced upon completion of the cell culture step b/. To this end, a person skilled in the art can empirically proceed, by testing several different doses of antigens and by determining the dose which, while being non-lethal for the cells, allows obtaining the highest production rate of the SLECs. For example, this production rate may be determined by the above-described Elispot test.

In particular implementations of the invention, step b/ of culturing the mononuclear cells is carried out for a period comprised between 36 and 60 hours, for example for a duration of about 48 hours. Advantageously, such a feature allows producing a large amount of SLECs while ensuring that the lifetime remaining to these SLECs upon completion of this culture step is as long as possible.

Step c/ of isolating the culture medium from the effector T cells that do not express at their surface the markers CD45RO and CD27 may be carried out in any manner known to a person skilled in the art. In particular, it may be carried out by collecting effector T cells rich in T cells specific to the antigen contained in the culture medium, then affinity chromatography using antibodies specific to the molecular markers CD45RO and CD27, the T cells that do not express at their surface the markers CD45RO and CD27 being present in the unbound fraction.

For example, the collection of the effector T cells rich in cells specific to the antigen, thanks to the prior antigenic stimulation, contained in the culture medium may be carried out by means of centrifugation at 3,000 rpm at 4° C. and then re-suspension of the pellet in saline phosphate buffer.

The next step of isolating the SLECs from the rest of the T cells, that is to say undifferentiated or non-totally differentiated T cells, advantageously takes advantage of the fact that human SLECs do not express at their surface the molecular markers CD45RO and CD27, glycoproteins that are expressed by the other types of human T cells, in particular by memory T cells. The implementation of an affinity chromatography step, by means of anti-CD45RO and anti-CD27 antibodies, immobilized on a solid support, advantageously allows rapidly and effectively separating the SLECs from the other lymphocyte subpopulations of the medium. The solid support implemented to this end may be of any conventional type. In particular, it may consist of magnetic beads on which these antibodies are grafted, implemented within a magnetic effect purification system such as the magnetic system commercialized by the company Miltenyi Biotec under the name MidiMACS®.

Preferably, all of the steps of the method are carried out in a sterile environment, and preferably at a temperature comprised between 18 and 20° C., with the exception of step b/ of cell culture, which is preferably carried out at 37° C.

The method according to the invention may further comprise a step of separating the antigen-specific SLECs contained in the obtained cell subpopulation.

Another aspect of the invention relates to an isolated cell subpopulation obtained by a method according to the invention, that is to say in the form of a purified subpopulation of T cells obtained in vitro or ex vivo, separated from the other T cell subpopulations by the previously-described isolation step, this subpopulation comprising short-lived human terminal effector T cells (SLECs) specific to the antigen linked to the disease of interest.

The cell subpopulation according to the production, predominantly comprising SLECs, may contain at least 50%, and even 60% (in number) of short-lived human terminal effector T cells specific to the antigen.

In particular embodiments of the invention, depending on the exact steps of the method having allowed obtaining it, and optional purification steps that it might include, the cell subpopulation according to the invention may contain at least 90%, in particular at least 95% (in number) of human terminal effector T cells with a short specific lifetime of the antigen, or consist of such antigen-specific cells linked to the disease of interest.

As indicated hereinabove, the terminal effector T cells SLECs are characterized by a phenotype CD45RA⁺/CD45RO⁻/CD27⁻ and a short lifetime, in the range of a few days, in particular less than or equal to five days. The cell subpopulation according to the invention, containing at least 50% of, or consisting of, antigen-specific SLECs, is particularly suitable for use as a medicine, in particular for the treatment of the disease of interest associated with the antigen against which these SLECs are specifically directed. When transferred to the subject who develops this disease, these antigen-specific SLECs effectively eradicate the pathological cells, and they disappear within 5 days post-administration, so that they do not trigger any rejection reaction by the organism.

In particular, the cell subpopulation obtained by the method according to the invention may be used for the treatment of a disease of interest of the infectious disease type, in particular of a viral pathology, such as influenza or an infection caused by a coronavirus, in particular by SARS-COV responsible for the severe acute respiratory syndrome (SARS) or by MERS-COV responsible for the potentially severe respiratory damage (MERS). In particular, this pathology may be Covid-19, caused by the SARS-COV-2 virus.

The cell subpopulation obtained by the method according to the invention may be used otherwise for the treatment of a disease of interest of the non-transmissible chronic disease type, such as cancer, multiple sclerosis, etc.

More generally, the cell subpopulation obtained by the method according to the invention may in particular be used for the treatment of any disease of interest to which an antigen, likely to cause the production by the human organism of specific T cells, may be associated.

In the present description, the term “treatment” means the achievement of a desired pharmacological and physiological effect. The term “treatment”, as used in the present description, includes the partial or total healing of the disease and/or the total or partial disappearance of one or more of its symptoms.

The cell subpopulation according to the invention may be administered to any subject in need thereof, that is to say affected or likely to be affected by the disease of interest. In particular, this subject may be a mammal, and in particular a human.

Preferably, the cell subpopulation according to the invention is administered to a subject needing it, that is to say affected or likely to be affected by the disease, less than 4 days, preferably less than 3 days and preferably less than 2 days, after production thereof, that is to say after the end of step b/ of cell culture of the method according to the invention implemented to obtain it.

The cell subpopulation of the invention is administered to the subject in a therapeutically-effective amount. By “therapeutically-effective amount”, it should be understood the amount sufficient to ensure the treatment of the disease. This amount depends on several factors, such as the disease and its gravity, the age, the weight, etc., of the subject to be treated, the administration route and form, etc. The therapeutically-effective amount of the cell subpopulation used according to the invention will be determined by the physician for each individual case.

The administration of the cell subpopulation according to the invention to the subject to be treated may be carried out by any conventional route known in the field of cell therapy, the parenteral routes, for example the subcutaneous route, intradermal route, subdural route, intravenous route, intramuscular route, intrathecal route, intraperitoneal route, intracerebral route, intra-arterial route or intra-lesional route, or the intranasal route, being particularly preferred in the context of the invention.

In particular, the systemic administration modes are preferred for the treatment of infectious diseases, in particular the modes of administration by injection, for example intravenous injection. For the treatment of non-transmissible chronic diseases, intra-tumor injection or mucosal administration, in particular intranasal, will be more particularly preferred.

The determination of the administration dosage of the cell subpopulation according to the invention is within the skills of the doctor. In particular, the cell subpopulation may be administered to the subject to be treated once, or several times, spaced apart by a few days to a few weeks.

The invention is also expressed in the terms of a method for the therapeutic treatment of a subject suffering from a disease of interest, in particular an infectious disease, in particular a viral disease, this method comprising a step of administering to this subject needing it a therapeutically-effective amount of an isolated cell subpopulation according to the invention, comprising short-lived terminal effector T cells specific to an antigen linked to this disease of interest. This method can address one or more of the characteristics described before with reference to the therapeutic use of the cell subpopulation according to the invention as a medicine.

The invention also relates to the use of a cell sub-population according to the invention for the manufacture of a medicament, in particular a medicine for treating infectious diseases, in particular viral diseases.

Another aspect of the invention relates to a pharmaceutical composition containing a cell subpopulation according to the invention, comprising short-lived terminal effector T cells specific for an antigen associated with a disease, as an active ingredient, in a pharmaceutically-acceptable carrier.

In the present description, by “pharmaceutically-acceptable carrier”, it should be understood any carrier useful for the preparation of a pharmaceutical composition and which is generally safe, non-toxic and neither biologically nor otherwise undesirable for the subject to be treated, in particular for mammals and especially humans.

The carrier of the pharmaceutical composition according to the invention may be liquid or semi-solid. It may be a diluent, an adjuvant or any other conventional carrier for the constitution of the pharmaceutical compositions, for example, an aqueous carrier, oily carrier, etc.

The pharmaceutical composition according to the invention may be in any galenic form, in particular in the form suitable for parenteral administration, in particular intravenous, or intranasal administration. As examples of such non-limiting dosage forms of the invention, mention may be made of the injectable suspension forms.

The pharmaceutical composition according to the invention may further contain one or several conventional excipient(s)/additive(s) for the constitution of the pharmaceutical compositions in the field of cell therapy, for example selected from among stabilizers, antioxidants, suspending agents, isotonic agents, surfactants, pH adjusters, buffering agents, etc., or any of their mixtures.

The pharmaceutical composition according to the invention may further contain one or several active ingredient(s) other than the SLECs specific to the antigen according to the invention, these active principles may act, or not, synergistically with these cells.

Preferably, the pharmaceutical composition according to the invention is formulated in the form of unit doses.

The invention also relates to the therapeutic use of a pharmaceutical composition according to the invention, as defined hereinbefore, for the treatment of a disease, in particular an infectious disease, in particular a viral disease. This use may respond to one or more of the characteristics described hereinbefore with reference to the therapeutic use of the cell subpopulation according to the invention.

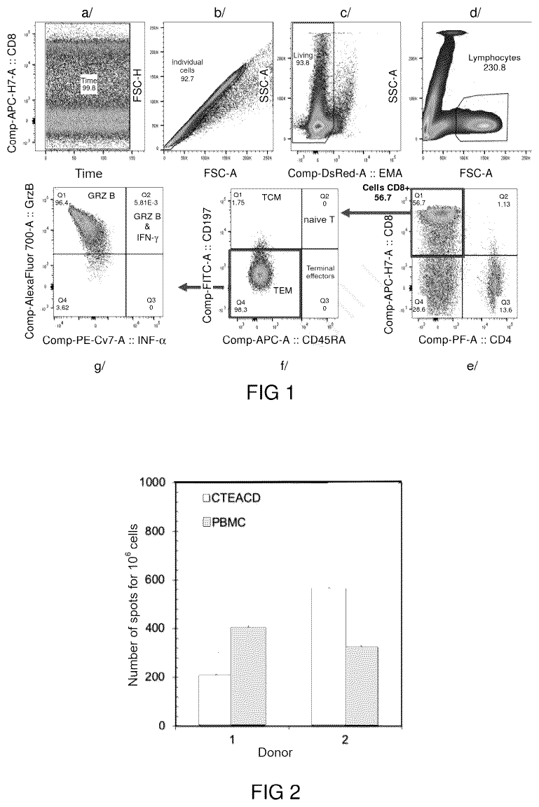

The features and advantages of the invention will appear more clearly in light of the examples of implementation hereinafter, provided for simple and in no way limiting illustration of the invention, with reference to FIGS. 1 to 5, wherein:

FIG. 1 shows flow cytometry images for human SLECs present in a population of human blood mononuclear cells (PBMCs) derived from an immunized individual having been infected with the influenza virus in the past. The PBMCs have been stimulated in culture with the influenza antigen (influenza vaccine killed by heat), then marked with the monoclonal antibodies directed against the indicated cell surface markers, cytokines and lytic molecules. The different windows respectively represent: a/ all of the human blood mononuclear cells (PBMCs); b/ the individual cells, not aggregated together; c/ the living cells; d/ the lymphocytes; e/ the CD8+ T lymphocytes; f/ the CD8+ effector T lymphocytes; g/ the CD8+ effector T lymphocytes which produce the lytic granules Granzyme B and IFN-γ (96.4%).

FIG. 2 represents a graph showing, for an ELISPOT immunoassay measuring the number of cells secreting IFN-gamma, the immunological characterization of the functionality of human blood mononuclear cells (PBMCs) and of human SLECs isolated from these PBMCs stimulated with the influenza antigen, derived from two different convalescent donor individuals having been infected with the influenza virus in the past (donor 1 and donor 2).

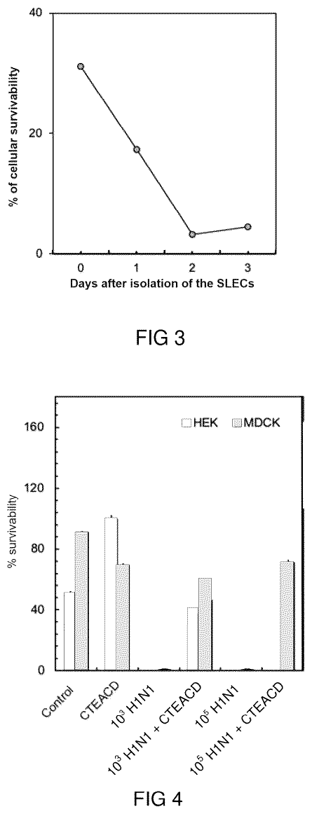

FIG. 3 represents a graph showing, over time, the % cell viability of human SLECs isolated from human blood mononuclear cells derived from an individual having been infected with the influenza virus in the past and having developed an immunity against this virus, stimulated with the influenza antigen, day 0 corresponds to the day of isolation of the SLECs after production thereof.

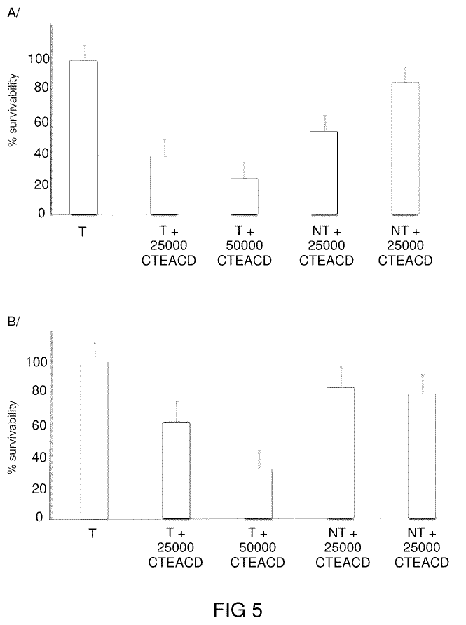

FIG. 4 represents a graph showing the % cell viability in vitro for HEK293 human cells (“HEK”) and MDCK canine cells (“MDCK”) infected with the H1N1 virus, 7 days after infection with 10³ or 10⁵ infectious particles, the cells having been cultured alone (“10³ H1N1”, “10⁵ H1N1”) or in the presence, as of 2 days after the infection, of human SLECs isolated from human blood mononuclear cells derived from an individual having been infected with the influenza virus in the past and having developed immunity against the virus, stimulated with the influenza antigen H1N1 (“10³ H1N1+SLEC”, “10⁵ H1N1+SLEC”); “Control” represents non-infected cells cultured alone and “SLEC” represents non-infected cells co-cultured with SLECs.

FIG. 5 represents graphs showing the % cell viability in vitro for HEK293 human cells in A/ and MDCK canine cells in B/, these cells having been transfected in order to express the antigens S, M, N and E of SARS-Cov2, the transfected cells having been cultured for 48 h alone (“T”) or in the presence, as of 2 days after transfection, of human SLECs isolated from human blood mononuclear cells derived from a Covid-19 convalescent individual (after in vitro culture and antigenic stimulation), at a rate of 25,000 SLECs (“T+25,000 SLEC”) or 50,000 SLECs (“T+50,000 SLEC”); “NT+25,000 SLEC” and “NT+50,000 SLEC” represents non-transfected cells cultured for 48 h in the presence of human SLECs isolated from human blood mononuclear cells derived from a Covid-19 convalescent individual (after in vitro culture and antigenic stimulation), at a rate of 25,000 SLECs and 50,000 SLECs, respectively.

### Example 1—Production and Isolation of a Cell Subpopulation Containing Human SLECs Specific for an Influenza Antigen

All of the steps of the method are carried out at 18 to 20° C., except for the cell culture step, and under aseptic conditions.

### 1.1/ Isolation and Stimulation of PBMCs From Human Peripheral Blood

### Equipment

30 ml samples (4×7.5 ml) of peripheral blood of two convalescent healthy human donors having been infected with the influenza virus in the past, in Vacutainer® sample tubes containing EDTA
UNI-SEP and UNI-SEPMAXI, UNI-SEP+ and UNI-SEPMAXI+ (Eurobio) tubes
Hanks (10×) buffer saline solution (Eurobio)
NaHCO₃ (Eurobio)
Fetal calf serum FCS (Eurobio)
PBS 1× saline phosphate buffer (Eurobio)
ethylenediaminetetraacetic acid EDTA from a stock solution at 500 mM, pH=7.8 sterilized in the autoclave
Erythrocyte lysis solution 10× (BD Biosciences)
Filtered trypan blue
Sterile distilled water
50 and 15 ml Falcon® sterile tubes (Dutcher France)
Tissue culture centrifuge Hettich® ROTINA 420/420R
AIM-V® cell culture medium (Gibco, Fisher)

### Isolation of the PBMCs

Human adult peripheral blood sampled in EDTA is used. 25 mL of blood are diluted with an equal volume of Hank 1× buffer+1% EDTA 0.5 M. The diluted blood is transferred into a UNI-SEP tube, which is closed and centrifuged (at 18-20° C.) at 3,000 rpm for 20 min (acceleration=9/deceleration=0). The tubes are removed from the centrifuge and transferred into a class II biological safety station. The layer of mononuclear cells is harvested using a pipette and the cell suspension is transferred into a new Falcon® tube. Ten ml of platelet-rich plasma are harvested in closed tubes and stored at −20° C. The suspension of mononuclear cells is diluted with a volume of Hank 1×+EDTA 5 mM and then centrifuged at 3,000 rpm, at 20° C. for 10 min (acceleration=9/deceleration=9). The supernatant is removed, and the PBMCs pellet is re-suspended in 20 ml of solution containing PBS 1× and 5 mM EDTA. The suspension is centrifuged at 3,000 rpm, at 20° C. for 10 min acceleration=9/deceleration=9). The supernatant is removed and the cell pellet, containing the PBMCs, is re-suspended in 10 ml of solution containing PBS 1× and 5 mM EDTA.

The concentration/amount of cells in the obtained cell suspension is determined by counting on a Malassez cell. To this end, 10 μl of cell suspension are mixed with 10 μL of previously filtered trypan blue, and the mixture is transferred into the Malassez chamber. The counting is carried out under a microscope over randomly selected 5 independent squares and the total is divided by 5. Since each square represents 0.01 μL, the number of cells per square is multiplied by 10⁵ to have the number of cells per mL according to the following formula:

Concentration(cells/ml)=(number of cells×10⁵)×2/5

Freezing of the cells is carried out as follows: centrifugation of the cell suspension at 3,000 rpm, 20° C. for 10 min (acceleration=9/deceleration=9); elimination of the supernatant; re-suspension in X mL of FCS medium (10 million cells in 2 mL of final volume) (X= 9/10th of the volume to have 5×10⁶ cells/ml− 1/10 of the volume is DMSO added dropwise to the cells under mild agitation). The cell suspensions are immediately cooled in ice before transfer to −80° C.

### Stimulation of the PBMCs

10 millions of recently isolated or thawed fresh PBMCs cells are introduced into a 15 ml polypropylene conical tube. The cells are centrifuged at 1,500 rpm, at room temperature, for 10 min and the supernatant decanted. The cells are re-suspended in 5 ml of AIM-VR (non-colored cells). 1 ml of cells are aliquoted in 3wells of a plate with 96 deep wells and 0.5 ml of cells are aliquoted in 4 wells of the plate. In each well: concanavalin A (Con-A at 2 μg/ml) or 15 μL of the antigens of the influenza vaccine Vaxigrip (influenza vaccine with injectable inactivated quadrivalent fragmented virion containing 2 type A strains (H1N1 and H3N2) and 2 type B strains (Yamagata and Victoria lines)), at a rate of 15 mg of hemagglutinin for each strain. The plate is placed in an incubator for culturing cells for 48-72 h at 37° C., 5% of CO₂. The cells are subsequently used for the purification of SLECs.

### 1.2/ Purification of Human SLECs

### Equipment

Magnetic microbeads grafted with an anti-CD45RO antibody (Miltenyi, ref. 130-046-001)
Magnetic microbeads grafted with an anti-CD27 antibody (Miltenyi, ref. 130-051-601)
Automated fluorescence cell counter LUNA-FL® (Dutcher)
5 ml sample tubes made of SIMPT415-6 polystyrene (VWR)
Separator MidiMACS® (Miltenyi Biotec)
Column LS for magnetic separation (Miltenyi Biotec, ref 130-042-401).

### Protocol

The number of cells in each well is determined using the fluorescence cell counter as described before. The cell suspension is centrifuged at 300 g for 10 min. The supernatant is completely aspirated and removed. The cell pellet is re-suspended in 80 μL of buffer A (solution containing saline phosphate buffer (PBS) at pH 7.2; 0.5% bovine serum albumin (BSA) and 2 mM EDTA) for 10⁷ cells. 20 μL of CD45RO microbead suspension are added for 10⁷ cells. The whole is mixed and incubated for 15 min at 4° C. After centrifugation at 300 g for 10 min, the cell pellet complexed with the microbeads is re-suspended in 2 ml of buffer A. The LS column is placed in the magnetic field of the MidiMACS® separator. It is prepared by rinsing with 3 mL of buffer A, then the cell suspension is deposited on the column. The non-marked cells that pass through the column are collected and the cells complexed with the microbeads retained by the column are washed with an appropriate amount of buffer A. The obtained fraction is named (−)CD45RO. Three steps of washing the column are carried out successively each by 3 ml of buffer A. The obtained fractions are successively named, respectively: (−)CD45RO/W1, (−)CD45RO/W2, (−)CD45RO/W3 (these fractions will allow recovering more cells (−)CD45RO if they have not been completely collected in the first fraction named (−)CD45RO. The column is removed from the separator and placed on a suitable sample tube. 3 ml of a suitable buffer (#130-091-222) delivered with the separation kit, are deposited on the column and then the magnetically marked cells are rapidly detached by pushing the piston firmly in the column. The obtained fraction is named (+)CD45RO and allows assessing the yield of the purification with the negative fraction.

A new column is prepared. The number of cells of the non-marked cell fraction (−)CD45RO is determined. This cell suspension (−)CD45RO is centrifuged at 300 g for 10 min. The supernatant is completely removed. The cell pellet is re-suspended in 80 μL of A buffer for 10⁷ cells (as indicated hereinabove). 20 μL of CD27 microbeads are added for 10⁷ cells. The whole is mixed and then incubated for 15 min at 4° C. The cells are washed by adding 2 ml of buffer A for 10⁷ cells, then, after centrifugation at 300 g for 10 min, the cell pellet is re-suspended in 2 ml of buffer A. It is a proceeded with the magnetic separation of the (+)CD27 cells and of the cells of the (−)CD27 fraction. To this end, the column is placed in the magnetic field of the MidiMACS® separator. The column is prepared by rinsing with 3 ml of buffer and then the cell suspension (−)CD45RO is applied on the column and the unfixed fraction named (−)CD45RO/(−)CD27 is collected. It corresponds to a population of SLECs containing SLECs specific for the influenza antigen. The column is washed with 3×3 mL of buffer and the fractions that are obtained successively are named, respectively: (−)CD45RO/(−)CD27/W1, (−)CD45RO/(−)CD27/W2, (−)CD45RO/(−)CD27/W3 (these fractions will allow recovering more cells (−)CD45RO/(−)CD27 if they were not completely collected in the first fraction named (−)CD45RO/(−)CD27)). The column is removed from the separator and placed on a suitable sample tube. 3 ml of buffer are deposited on the column, then the magnetically marked cells are rinsed rapidly by pushing the piston firmly in the column. The obtained fraction is named (−)CD45RO/(+)CD27.

### 1.3/ Analysis of Human SLECs

### Flow Cytometry Analysis

The protocol of the experiment is as follows. Polychromatic flow cytometry is carried out using a BD® LSR-II cytometer having 3 laser beams and the analysis of the data obtained after acquisition is carried out with the software FlowJo. The PBMCs cells are cultured in serum-free AIM5 culture medium in deep well 96-well plates at a density of 2×10⁶ per mL, and then stimulated with the antigens as indicated for ELISPOT. The cells are incubated for 5 days at 37° C., 5% CO₂ and saturating moisture in a cell culture incubator. The cells are restimulated for 6 h in the presence of the CD28 co-stimulatory antibodies (BD, Clone CD28.2) and CD49d (BD, Clone 9F10) (BD Biosciences, France) and Brefeldine A (Invitrogen® 00-4506-51). Afterwards, the cells are rinsed with 1×PBS+1% BSA and incubated for 30 min at room temperature with a combination of cell surface marking antibodies (Pacific Blue® anti-CD3 (BD, Clone SP34-2), APC-H7anti-CD8 (SK1), PE anti-CD4 (BD, Clone L200), APC anti-CD45RA (BD, Clone 5H9), FITC anti-CD197 (BD, Clone 150503). Ethidium monoazide (EMA) at a rate of 0.5 mg per well is also added to enable detection and exclusion of dead cells. After incubation for 15 min at room temperature, the cells are rinsed with 1×PBS+% BSA, then the cells are fixed and permeabilized by incubation for 15 min at room temperature in 2 mL of the BD Cytofix/Cytoperm® solution diluted at 1×. The cells are rinsed 5 min with the solution Perm/Wash of BD and then the intracellular markings are carried out with the monoclonal antibodies PE-Cy® 7 anti-IFN-γ (BD, Clone B27) and Alexa Fluor® 700 anti-Granzyme B (BD, Clone GB11). After incubation for 30 min at room temperature, the cells are rinsed with 1×PBS+% BSA, then the re-suspended cell pellets in 250 mL of 1% paraformaldehyde in 1×PBS and then stored in the dark at 4° C. until acquisition by the LSR-II. The cells are acquired with the flow cytometer and selected according to their particle grain distribution and size (pro-diffusion and lateral diffusion) corresponding to the lymphocytes (FSC/SSC), then to the EMA−, CD3+ populations, high CD4+ and high CD8+ (>100,000 events). The result of the flow cytometry analysis is shown in FIG. 1. It is observed that the CD8+ T cells of the human SLECs present in the PBMCs cells of the subject having been infected by influenza and vaccinated against influenza repeatedly, and which have been stimulated in culture with the antigen, are composed of more than 98% of effector cells. More than 96% of the SLECs CD8+ T cells are Granzyme B+ and 40% also secrete IFN-gamma.

### Elispot Analysis

The protocol of the experiment is carried out as follows. Two hundred fifty thousand PBMCs or SLECs [(−)CD45RO/(−)CD27] of donors 1 and 2 are inoculated with triplicate in wells of a 96-well plate of a human IFN-gamma ELISA kit (Mabtech) in a final volume of 200 mL, then stimulated with 15 mL of the antigens contained in the influenza vaccine VaxiGripTetra, overnight in a cell culture incubator. Negative controls (cells not stimulated with the antigen) and positive controls (cells stimulated with ConA) are used in parallel. Following an incubation night, the cells are removed from each well and the detection of the specific spots of IFN-gamma is carried out according to the protocol of the Mabtech kit. The secondary antibody (human 7-B6-1 biotinylated, anti-INF-γ) is diluted to 1/1,000 in 1×PBS with Ca²⁺ and Mg²⁺ and supplemented with 0.5% of FCS. In parallel, the Streptavidin-ALP (alkaline phosphatase) substrate is also diluted to 1/1,000 in 1×PBS with Ca²⁺ and Mg²⁺ and supplemented with 0.5% of FCS. The plates no longer containing cells are rinsed 5 times with 200 μl PBS×1 per well, then drained on a clean absorbent paper. 100 μl of diluted antibody are added in each well and the plate is incubated at room temperature for 2 h. Afterwards, the solution is removed, the wells rinsed 5 times with the buffer PBS×1 and then 100 μl of Streptavidin-ALP diluted to 1/1,000 are added and the plates incubated for 1 h at room temperature. Afterwards, this solution is removed, the plates rinsed 5 times with PBS×1 before adding 100 μl of the BCIP/NBT substrate (0.4 μm). The plates are incubated at room temperature in the dark until the apparition of the spots (about 12-15 min). The plates are immediately rinsed with tap water, drained and then allowed to dry completely before counting the spots. Each spot is considered to originate from an IFN-gamma secretory cell specific to the antigens.

The obtained results, in terms of the number of cells secreting IFN-gamma, are shown in FIG. 2, for each of the two donor individuals, for PBMCs and for SLECs. It is observed that the in vitro stimulation of the SLECs enables a massive secretion of IFN-gamma. The mononuclear cells (PBMCs) of the donor 2 have a higher proportion of T cells specific to the influenza antigen which are more reactive and which are differentiated more strongly into terminal effector T cells (SLECs). These results clearly demonstrate that the reactive cells in the PBMCs of the donor 2 are more predominant than in the PBMCs of the donor 1.

All of the results hereinabove demonstrate that the SLECs have all of the characteristics of cytotoxic effector cells armed to eradicate the cells infected by influenza.

### Cytotoxicity Test

SLECs (10⁶ cells (−)CD45RO/(−)CD27/well) are cultured in suspension in an RPMI medium supplemented with 10% fetal calf serum in wells of a 12-well plate, then incubated in a cell culture incubator (37° C., 5% CO₂ and saturating moisture). At different post-incubation times, fractions (20 ml) are sampled for the measurement of cell viability. The cell viability is measured by means of a Luna® cell counter, for three days after the end of the purification step. The day 0 corresponds to the day of isolation and culturing of the SLECs.

The obtained results are shown in FIG. 3. It is observed that the lifetime of the SLECs does not exceed 3 days in cell culture.

### Example 2

### A/ In Vitro Study of the Capacity of Human SLECs Specific for an Influenza Antigen to Eliminate Human or Canine Cells Infected With Influenza

The main objective of this experiment is to assess the effectiveness of the SLECs isolated from the blood of a convalescent donor having been infected with the influenza virus and having been vaccinated against this virus in the past, recognizing and cytolyzing the cells infected with the H1N1 virus in vitro.

The human cells HEK-293 and the canine cells MDCK, known for their susceptibility to influenza viruses, are used for this experiment.

These cells cultured in the DMEM medium with 10% fetal calf serum are seeded in 24-well plates at the rate of 10⁴ cells per well, and in 6-well plates at the rate of 10⁶ cells per well. The monolayer of cells are inoculated with the H1N1 virus (with 103 (for the 24-well plate cells) and 10⁵ (for the 6-well plate cells) infectious particles of the virus), at a multiplicity of infection of 0.1, then they are either maintained as they are, or co-cultured 48 h after infection with SLECs (−)CD45RO/(−)CD27 prepared as indicated hereinabove.

The cells are analyzed after 5 days of culture using the Abcam ab112118 cytotoxicity test kit, in order to determine the cell viability rate.

Non-infected cell controls (“control”) and non-infected cells co-cultured with the SLECs (“SLEC”) are also carried out.

The obtained results are shown in FIG. 4. They demonstrate that all of the infected cells are cytolysed by the viral infection, while there is no cytolysis of the uninfected cells used as controls. The infected cells co-cultured with the SLECs have been protected from viral diffusion and have proliferated. This is because the SLECs rapidly lyse the cells infected with the virus and protect the non-infected cells from death by re-infection with the virus.

### B/ In Vitro Study of the Capacity of Human SLECs Specific for SARS-COV-2 Antigens to Eliminate Human or Canine Cells Transfected With Plasmids Expressing the Genes With the Structure S, M, N and E of Coronavirus

The four recombinant plasmids expressing the S, M, N and E genes of SARS-CoV-2, respectively, have been obtained for free from BEI, USA (BEI references: NR-52394, NR-53508, NR-52973 and NR-52967).

These plasmids are introduced into the bacterial strain E coli JM109 competent by conventional transformation using the protocol provided by Promega. The bacteria carrying the plasmids are selected by culture in a selective medium containing 100 mg/ml of ampicillin. The recombinant bacteria carrying the plasmids are amplified in liquid LB medium cultures (1 L) supplemented with 100 mg/ml of ampicillin and cultured for 24 h at 32° C. with stirring. The extraction of plasmid DNA is carried out using a Maxprep kit (Macherey Nagel) and the exact protocol delivered with the kit. The concentration of the isolated DNAs is determined by spectrophotometric reading at the wavelength of 260 nm. The DNAs are transfected into HEK-293 and MDCK cells using a commercial kit (TransIT®, Mirus/Euromedex) and the protocol of the MIR 5404 kit. A DNA mixture containing 1.25 mg of each of the 4 plasmids is used for each transfection. At 24 h post-transfection, the transfected cells are dissociated and inoculated at a rate of 200,000 cells per well in triplicate in a plate of 24 cell culture wells. 24 h later, the cell triplicates (HEK-293 and MDCK) are co-cultured either with 25,000 SLECs [(−)CD45RO/(−)CD27] or with 50,000 SLECs [(−)CD45RO/(−)CD27] isolated (as described hereinabove) from the peripheral blood of a donor convalescent of Covid-19 since 4 months. This donor was such that the mononuclear cells contained in his peripheral blood have a very high proportion of specific cells of SARS-COV-2 which secrete IFN-γ (>3,500/10⁶ PBMCs), determined by an ELISPOT test. After 48 h of coculture, the cells are examined for the survival and cytolysis proportions. The results of this analysis are reported in FIG. 5. These results confirm the high cytolysis efficacy of the cells expressing the SARS-COV-2 antigens by the anti-Covid SLECs.

### Example 3—In Vivo Study of the Capacity of the Murine SLECs Specific for an Influenza Antigen to Treat Mice Infected by Influenza

### 3.1/ Production and Isolation of SLECs

SLECs have been produced from mouse spleen cells, according to the following protocol.

### Equipment

Automated fluorescence cell counter LUNA-FL® (Dutcher)
Flow cytometer BD® LSR-II
5 ml sample tubes made of SIMPT415-6 polystyrene (VWR)
RPMI Medium (GIBCO, ref 21875-034)
FCS (Eurobio)
PBS 1× (Eurobio)
EDTA from a 500 mM stock solution
Lysis solution (BD Biosciences, ref 555 899)
Sterilized blade and lamellae
Falcon® 15 ml tubes
Concanavalin A (ConA) (Thermo Fisher, ref 00-4978-03)
VaxigripTetra (Sanofi Pasteur) vaccine
Recombinant structural proteins of SARS-COV-2 (BEI, USA)
Anti-biotin magnetic microbeads (Miltenyi Biotec, ref 130-090-485)
Anti-KLRG1-biotin antibodies, mice, REA1016, (Miltenyi Biotec ref. 130-117-096)
Anti-CD27-Biotin antibody, human and mouse, REA499, (Miltenyi Biotec ref. 130-114-164)
MidiMACS® separator (Miltenyi Biotec ref. 130-042-302)
Column LS for magnetic separation (Miltenyi Biotec, ref. 130-042-401).

### Mouse and Splenocyte Blood Harvesting

Mice (2 groups of 4 mice) C57BI6 aged 8 weeks originating from Janvier establishments and housed in the animal complex of LBFA/UGA are immunized by intramuscular injection with 1/100th (0.5 ml of the vaccine in 49.5 ml of PBS×1) of a human vaccine dose of the commercial vaccine VaxigripTetra for the group 1. The mice of group 2 are immunized with a mixture of the 4 structural proteins (S, M, N and E) at a rate of 0.5 mg of each of the proteins in a final volume of 100 ml of 1×PBS. At three weeks post-immunization, the mice are put into deep sleep (anesthesia) with isoflurane vapor. After incision of the abdominal cavity, the spleens of the mice are isolated aseptically and directly transferred into the Falcon® tubes containing 7 ml of RPMI medium and stored in ice. Afterwards, the spleens are placed between a sterile blade/lamella and then macerated. The splenocytes are collected in 5 ml of RPMI medium supplemented with 10% FCS. After centrifugation for 10 min at 300 g at 4° C., the splenocytes are washed with 5 mL of PBS 1×+5 mM ETDA solution. After centrifugation for 10 min at 300 rpm at 4° C., the splenocytes are treated for 2 min with an erythrocyte lysis solution (BD Biosciences) to remove the red blood cells. After centrifugation for 5 min at 300 g at 4° C., the cells are counted in the presence of trypan blue using the LUNA-FL® cell counter.

### Splenocyte Stimulation

ConA is used to stimulate the proliferation of the splenocytes, as a positive control.

The vaccine VaxigripTetra, containing influenza antigens, is used to stimulate the differentiation of T cells into SLECs. 3 μL of a solution of ConA (in PBS×1) at 1 μg·mL⁻¹, 15 μL of antigenic vaccine solution VaxigripTetra or 2 mL of the mixture (S+M+N+E) of SARS-COV-2 structural recombinant proteins are added in 3 mL of a cell suspension (2×10⁷ cells/mL) in RPMI+10% FCS. The suspensions of cells are transferred into tubes made of polystyrene and incubated for 3 days at 37° C. in a cell culture incubator under a CO₂ atmosphere.

### Isolation of the SLECs

The number of cells is determined using the fluorescence cell counter. After centrifugation of the cell suspension at 300 g for 10 min, the supernatant is removed completely. Up to 10⁷ nucleated cells are re-suspended by 45 μL of buffer (PBS pH 7.2+0.5% of bovine serum albumin+2 mM EDTA). 5 μL of the anti-KLRG1-Biotin antibody are added, the mixture is mixed and incubated for 10 min in the dark at 4° C. The cells are washed by adding 2 ml of buffer and centrifuged to 300 g for 10 min. The supernatant is completely aspirated. This washing step is repeated. The cell pellet is re-suspended in 80 μL of buffer. 20 μL of anti-biotin magnetic microbeads are added for 10⁷ cells. After mixing, the whole is incubated for 15 min at 4° C., then the cells are washed by adding 2 ml of buffer for 10⁷ cells and centrifuged at 300 g for 10 min. The supernatant is completely aspirated and the pellet is re-suspended with 2 ml of buffer. The LS column is placed in the magnetic field of the MidiMACS® separator and prepared by rinsing with 3 ml of buffer. The 2 ml of cell suspension is applied to the column. The unbound cell fraction, named (+) KLRG1, is collected.

This cell suspension is centrifuged at 300 g for 10 min. The supernatant is completely aspirated and the cell pellet is re-suspended, at a rate of up to 10⁷ nucleated cells per 100 μL of buffer. 10 μL of the mouse/human anti-CD27 antibody are added, the whole is mixed and incubated for 10 min at 4° C. in the dark. The cells are washed by adding 2 ml of buffer and centrifuged at 300 g for 10 min. The supernatant is completely aspirated and the washing step is repeated. The cell pellet is re-suspended in 80 μL of buffer, and 20 μL of anti-biotin magnetic microbeads are added for 10⁷ cells. After mixing, the whole is incubated for 15 min at 4° C. The cells have been washed by adding 2 ml of buffer for 10⁷ cells and centrifuged at 300 g for 10 min. The supernatant is completely aspirated and the cell pellet is re-suspended in 2 mL of buffer. The LS column is placed in the magnetic field of the MidiMACS® separator. The LS column is prepared by rinsing it with 3 mL of buffer. The cell suspension is applied on the column and the unbound cell fraction is collected. It contains SLECs (+)KLRG1/(−)CD27 cells.

### 3.2/ Therapeutic Efficacy

4 groups of BALB/c mice have been infected with the H1N1 influenza virus (20,000 infectious particles in 20 ml) by the intra-nasal route.

A group (Group 1) has been kept as such. On day 2 post-infection, SLECs [(+)KLRG1/(−)CD27] derived from C57BL/6 mice not immunized with VaxiGripTetra (Group 2, 10⁵ SLECs/mice) have been transferred, by intraperitoneal injection, to one group. In parallel, SLECs [(+)KLRG1/(−)CD27] derived from C57BL/6 mice having been immunized beforehand against the influenza as described hereinabove, have been transferred, by intraperitoneal injection, to two other groups. (Group 3, 5×10⁴ SLECs/mice and Group 4, 2.5×10⁵ SLECs/mice).

After 4 to 5 days, in Group 1, 3 mice out of 7 are dead and the 4 others, in a very deteriorated state due to the H1N1 infection, have been euthanized. The health condition of the mice of Group 2 to which normal mouse SLECs have been administered has deteriorated 5 to 6 days after infection with H1N1. These diseased mice have been euthanized on day 6 post-infection. Conversely, all of the mice of Group 3 and Group 4 have remained alive for more than 6 months without showing any clinical signs.

These results give the clear demonstration that the adoptive transfer of SLECs from immunized mice is associated with a persistent curative therapy in all of the treated animals (14/14).

It is further demonstrated that the transfer of SLECs isolated from C57BL/6 mice in BALB/c mice of different genetic background does not induce any sign of rejection disease or of reaction of the graft against the host after more than 3 months of observation.

All of the examples hereinabove provide evidence of the capacity of the SLECs produced in accordance with the invention to eliminate infected cells, and consequently to prevent the development of the disease, both in culture of human or canine cells in vitro, and in vivo in mice.

Furthermore, the BALB/c mice which have been infected with H1N1 and treated with the anti-H1N1 SLECs have been monitored for more than 10 months without it being observed with a pathological sign or an apparent secondary effect.

Following euthanasia, anatomopathological observations have not allowed detecting any lesion in the observed organs (lung, spleen, liver, heart). These results demonstrate that the treatments with the SLECs are not associated with side effects even after a long post-treatment period.

### 3.3/ Repeated Injections

In order to assess whether the repeated therapeutic use of SLECs could cause, or not, a development of disease, 6 groups of 6 BALB/6 mice aged 8 weeks have been inoculated with 250,000 SLECs derived from C57 BL/6 mice immunized against influenza as described hereinabove. The mice of group 1 have been kept as controls, the mice of group 2 have received 2 injections with an interval of 10 days, the mice of groups 3, 4, 5 and 6 have respectively received 3, 4, 5 and 6 injections at intervals of 10 days. The animals have been kept under observation at least 3 months after the last injection. The results showed no anomaly or disturbance during monitoring. Indeed, no loss of weight, appetite or hair has been observed. All of the animals have remained active and have shown no difference with the group of control mice.

### Example 4—In Vivo Study of the Capacity of the Murine SLECs Specific for a SARS-COV-2 Antigen to Treat Mice Infected With SARS-COV-2

BALB/c mice have immunized against SARS-COV-2 with a vaccine composed of inactivated SARS-COV-2 virus (Obtained from BEI Ressources, USA) killed by heat (65° C., 30 min). Each mouse has received by the intramuscular route 50 ml of the vaccine diluted 50 times in the saline solution (1×PBS), then a boost under the same conditions 2 weeks later. The splenocytes have been isolated from mice immunized 2 months after boost and cultured for 48 h in the presence of SARS-COV-2 antigens (obtained from BEI Ressources, USA). The SLECs have then been isolated and used in anti-Covid therapy in infected mice.

In the absence of the existence of a natural mouse model for SARS-COV-2, the transgenic mouse model K18 hACE-2 has been used. These mice express in all of the cells of the organism the human receptor hACE-2 and become susceptible to infection by the virus.

Two groups of eight K18 hACE-2 mice have been infected with the delta strain of the SARS-COV-2 virus (5×10³ mouse infectious particles) by the intra-nasal route and then one of the two groups has been treated with about 50,000 SLECs obtained as described hereinabove by the intravenous route. 2 control mice have received PBS with no virus. At 6 days, all of the untreated infected animals and 2 animals of the treated group have been euthanized because of the deterioration of their health condition. At least 60% of the mice infected and treated with the SLECs survived on day 7 and 40% on day 8 (the last day of the protocol).

These results demonstrate that despite the absence of a suitable murine model and the limitations of the murine model K18, the treatment with SLECs has demonstrated a partial protection of the treated animals.

### Example 5—Production of Mononuclear Cells Comprising T Cells Specific for an Antigen With a High Proliferative Capacity From Cord Blood

The first step consists in generating adherent antigen-presenting cells (APCs) and then loading them with the antigens so that they present them via the MHC and finally putting them in co-culture with the non-adherent cells containing the T lymphocytes to render them specific to the antigen.

### Generation of the APC Loaded With the Antigen

The mononuclear cells of the cord blood have been purchased from Stemcell Technologies. The cell culture media have been purchased from Eurobio and from Fisher Technologies.

On the first day, an RPMI 1640 medium supplemented with 10% fetal calf serum (FCS) (R10 medium) is preheated to 37° C. in a water bath. The mononuclear cells of the cord blood are extracted from the liquid nitrogen and incubated in the ice and then at 37° C. in the water bath until the cell suspension is semi-molten. Afterwards, the cell suspension is transferred dropwise into a 50 mL sterile conical tube containing 10 ml of preheated medium. Afterwards, the suspension is homogenized and centrifuged at 1,500 rpm for 10 min at room temperature.

The cell pellet is re-suspended in a medium R10 at 2×10⁶/ml and then seeded in the wells of a 12-well plate at a rate of 1 ml/well and incubated for 4 h at 37° C. under cell culture conditions.

The non-adherent cells that have remained in suspension are harvested and cryopreserved in a medium composed of 90% of FCS supplemented with 10% DMSO.

The plastic adherent cells are cultured in 1 mL of R10 medium supplemented with GM-CSF cytokine (800 LJ/mL) and IL-4 (500 U/mL). After 3 days of culture, half (0.5 mL) of the medium is replaced with fresh R10 medium containing GM-CSF and IL-4. On day 6 of the culture, half (0.5 mL) of the medium is replaced with a medium containing 500 U of IL-4 and 60 ng of TNF-α. On day 9, the antigen (influenza or inactivated SARS-COV-2) is added at a rate of 1 ug/mL and then the cells are incubated for 18 to 24 h before removing the medium and rinsing the cells with fresh R10 medium.

### Co-Culture With Non-Adherent Cells

The non-adherent cells are removed from the liquid nitrogen, incubated in the ice and then at 37° C. in order to thaw them as described hereinabove. The cell pellet is re-suspended in R10 medium at a rate of 10⁶ cells/mL and then incubated for 4 h at 37° C. Finally, 1 mL of cell suspension is seeded on the monolayers of adherent cells by replacing their medium culture. The co-culture is incubated for 3 days and then supplemented with IL-2 in an amount of 5 U/mL and then incubated for 6 more days.

This method allows deriving antigen-presenting cells from cord blood and charge them with antigens.

The non-adherent cells obtained upon completion of the coculture step, comprising a population of mononuclear cells comprising T cells specific to the antigen with a high proliferative capacity, may be used for steps bland c/ of a method according to the invention.

1. An in vitro or ex vivo method for producing and isolating a cell subpopulation comprising T cells specific for an antigen linked to a disease of interest, wherein it includes steps of:
a/ starting from an isolated human biological sample, obtaining a population of mononuclear cells comprising T cells specific for the antigen with a high proliferative capacity,
b/ culturing the mononuclear cells for 24 to 72 hours in a suitable cell culture medium containing the antigen,
and c/ isolating from the culture medium the T cell that do not express at their surface the markers CD45RO and CD27.

2. The method according to claim 1, wherein step a/ of obtaining a population of mononuclear cells comprising T cells specific for the antigen with a high proliferative capacity comprises identifying a human peripheral blood biological sample comprising a population of T cells specific for the antigen with a high proliferative capacity, and isolating mononuclear cells from the biological sample.

3. The method according to claim 2, according to which the human peripheral blood biological sample is a peripheral blood sample of a human individual having been affected by the disease.

4. The method according to claim 1, according to which step b/ of culturing the mononuclear cells is carried out for 36 to 60 hours.

5. The method according to claim 1, according to which step c/ of isolating from the culture medium the T cells that do not express at their surface the markers CD45RO and CD27 is carried out by collecting the cells contained in the culture medium, and then affinity chromatography by means of antibodies specific for the molecular markers CD45RO and CD27, said the T cells which do not express at their surface the markers CD45RO and CD27 being present in the unbound fraction.

6. An isolated cell subpopulation obtained by a method according to claim 1, comprising short-lived human terminal effector T cells specific for the antigen linked to the disease of interest.

7. The cell subpopulation according to claim 6, containing at least 50% of short-lived human terminal effector T cells specific for the antigen.

8. The cell subpopulation according to claim 6, containing at least 90% of short-lived human terminal effector T cells specific for said the antigen.

9. The cell subpopulation according to claim 6, consisting of short-lived human terminal effector T cells specific for the antigen.

10. The cell subpopulation according to claim 6, for use thereof as a medicine.

11. The cell subpopulation for use thereof according to claim 10, for the treatment of the disease of interest.

12. The cell subpopulation obtained by a method according to claim 1, comprising short-lived human terminal effector T cells specific for the antigen linked to the disease of interest for use thereof as a medicine, according to which the cell subpopulation is administered to a subject needing it less than 4 days after the end of the cell culture step b/ of the method implemented in order to obtain it.

13. The cell subpopulation for use thereof according to claim 10, for the treatment of an infectious disease.

14. The cell subpopulation for use thereof according to claim 10, for the treatment of a non-transmissible chronic disease.

15. A pharmaceutical composition wherein it contains a cell subpopulation according to claim 6 in a pharmaceutically-acceptable carrier.

16. The pharmaceutical composition according to claim 15, in a form suitable for parenteral administration.
